(19) United States
(12) Patent Application Publication
(10) Pub. No.: US 2003/0024856 A1
(43) Pub. Date: Feb. 6, 2003

(54) Method for removal of oder from poly alpha-olefins

(76) Inventors: Phil Surana, Somerset, NJ (US); Norman Yang, Westfield, NJ (US)

Correspondence Address: ExxonMobil Chemical Company, Law Technology, P.O. Box 2149, Baytown, TX, 77522-2149, US

(21) Appl. No.: 09/817,379

(22) Filed: Mar. 26, 2001

(51) Int. Cl. C07C 7/12; C10M 175/00; C10G 25/00

(52) U.S. Cl. 208/299; 208/182; 585/820; 585/829

(57) ABSTRACT

This invention provides a facile method for removing odor-causing species from lubricants. In this method, such species are removed by contacting the lubricant composition with at least one zeolite. Preferred zeolites include the type 13X Molecular Sieve, i.e., Na₈₆[(AlO₂)₈₆(SiO₂)₁₀₆].276 H₂O.

### FIELD OF THE INVENTION

This invention belongs to the field of synthetic lubricants. More particularly, this invention relates to a method for removing odor from synthetic lubricants derived from poly α-olefins.

### BACKGROUND OF THE INVENTION

Poly α-olefins comprise one class of synthetic hydrocarbon lubricants which have achieved importance in the lubricating oil market. These materials are typically produced by the polymerization (the term “oligomerization” is often use for the lower molecular weight products which are used as low viscosity basestocks) of α-olefins typically ranging from 1-octene to 1-dodecene, with 1-decene being a preferred material, although polymers of lower olefins such as ethylene and propylene may also be used, including copolymers of ethylene with higher olefins, as described in U.S. Pat. No. 4,956,122 and the patents referred to therein. The poly α-olefin (PAO) products may be obtained with a wide range of viscosities varying from highly mobile fluids of about 2 cSt at 100° C. to higher molecular weight, viscous materials which have viscosities exceeding 100 cSt at 100° C. The PAO's are conventionally produced by the polymerization of olefin feed in the presence of a catalyst such as AlCl₃, BF₃, or BF₃ complexes. Processes for the production of PAO lubricants are disclosed, for example, in the following patents: U.S. Pat. Nos. 3,382,291; 4,172,855; 3,742,082; 3,780,128; 3,149,178; and 4,956,122. The PAO lubricants are also discussed in Lubrication Fundamentals, J. G. Wills, Marcel Dekker Inc., (New York, 1980). Subsequent to the polymerization, the lubricant range products are hydrogenated in order to reduce the residual unsaturation. In the course of this reaction, the bromine number of the lubricant is reduced from typical values of about or higher for low viscosity PAO's and 5 to 15 for high viscosity PAO's to a value of not more than about 2 or even lower.

Low viscosity PAO's (i.e., 2-10 cSt at 100° C.) generally contain odor-causing species which are believed to be low boiling oxygenates. This odor problem in low viscosity PAO's renders these lubricants unacceptable for use in many applications. A typical method for removal of such odor-causing species is steam distillation.

### SUMMARY OF THE INVENTION

This invention provides a facile method for removing odor-causing species from lubricants. In this method, such species are removed by contacting the lubricant composition with at least one zeolite. Preferred zeolites include the type 13X Molecular Sieve, such as that sold by UOP, Inc., and W. R. Grace, i.e., Na₈₆[(AlO₂)₈₆(SiO₂)₁₀₆].276 H₂O, as well as the type 4A Molecular Sieve, Na₁₂(AlO₂)₁₂(SiO₂)₁₂.27 H₂O

### DETAILED DESCRIPTION OF THE INVENTION

The present invention provides a method for removing odor-causing species from a lubricant composition, which comprises contacting said composition with at least one zeolite.

In the practice of the invention, it is preferred that the zeolite utilized is a Molecular Sieve of Type 4A or 13X. Especially preferred is Molecular Sieve of Type 13X, in the form of beads of clay/zeolite blends of a diameter of 1/16 to ⅛ inch. In the examples below, a bed of Molecular Sieves of diameter of 1/16 inch were placed in a glass column of a diameter of about 1 inch, and the lubricant composition fed to the bottom of the column under sufficient pressure to move the material to the top of the column where it was removed. While the present invention contemplates the feeding of the lubricant composition to either the top or bottom of such a column, it is preferred that it be fed from the bottom in order to minimize or eliminate channeling within the packed Molecular Sieves.

It is further preferred that the lubricant composition be contacted with such zeolites at a temperature of about 40° F. to 130° F., most preferably 70° F. to 100° F., and at pressures of about 0 psig to 5.0 psig, most preferably 0 psig to 2.0 psig.

In a further aspect of the invention, there is provided a method for removing odor-causing species from a lubricant composition which comprises feeding said lubricant to a column packed with Type 13X Molecular Sieve or Type 4A Molecular Sieve, preferably Type 13X, at a temperature of about 40 to 130° F., preferably about 70° F. to 100° F., at a pressure of about 0 psig to 5.0 psig, preferably about 0 psig to 3.0 psig, for an average residence time of about 0.15 hours to 3.5 hours. In a preferred embodiment, the lubricant composition is comprised of at least one poly α-olefin derived lubricant having a viscosity of from about 2-10 cSt at 100° C. In a further preferred embodiment, the temperature is about 70° F. to about 95° F., and the pressure is about 0 psig to about 2.0 psig. In the practice of this aspect of the invention, it is further preferred that the average residence time in said column is about 1.0 hour to about 3.5 hours, most preferably about 1.5 hours to about 2.0 hours.

Once utilized according to the method of the present invention, the Molecular Sieves may be regenerated by utilizing the following procedure which was utilized in the examples below:

1. The Molecular Sieves are regenerated at 660 F for 2.5 hours at lmm Hg absolute pressure.

2. A N₂ purge at 5 cc/minute (100 cc bed) is sent through the bed for 30 minutes at 660° F.

3. The heat is turned off and N₂ purge continued to cool the bed to room temperature.

4. The amount of oil recovered from the sieves during regeneration is ˜20 grams.

### EXPERIMENTAL SECTION

### Example 1

2 cSt PAO was treated over a 100 cc volume of 13X molecular sieve bed, packed in a glass column, at temperatures from 50° F. to 95° F. and at liquid hourly space velocities (LHSV's) ranging from 3.0 to 6.0 hours. Table-1 compares molecular sieve treatment to the industry accepted steam distillation for odor removal.

TABLE 1

| Product | Treatment | Odor Level |
|---|---|---|
| 2 cSt PAO | None | 5 (strong odor) |
| 2 cSt PAO | Steam distillation | 0 (No odor) |
| 2 cSt PAO | Molecular Sieve | 0 (No odor) |

Table-2 compares the effect of residence time on the adsorption capacity of molecular sieves. The longer the residence time the higher the capacity in grams of oil processed with zero odor.

TABLE 2

| | LHSV | | Odor | Bed Life: Grams |
|---|---|---|---|---|
| Product | (hours) | Temperature | Level | Of Oil Processed |
| 2 cSt PAO | 6.0 | 77° F. | 0 | 6029 |
| 2 cSt PAO | 3.0 | 77° F. | 0 | 8816 |

### Example 2

4 cSt PAO was treated over a 100 cc volume of 13X molecular sieve bed, packed in a glass column, at temperatures from 50° F. to 95° F. and at liquid hourly space velocities ranging from 1.8 to 3.0 hours. Table-3 compares molecular sieve treatment to the industry accepted steam distillation for odor removal

TABLE 3

| Product | Treatment | Odor Level |
|---|---|---|
| 4 cSt PAO | None | 5 (strong odor) |
| 4 cSt PAO | Steam distillation | 0 (No odor) |
| 4 cSt PAO | Molecular Sieve | 0 (No odor) |

Table-4 compares the adsorption capacity of 13X molecular sieves as a function of residence time for 4 cSt PAO. The adsorption capacity increases with the higher residence time.

TABLE 4

| | LHSV | | Odor | Bed Life: Grams |
|---|---|---|---|---|
| Product | hours | Temperature | Level | Of Oil Processed |
| 2 cSt PAO | 6.0 | 77° F. | 0 | 6029 |
| 2 cSt PAO | 3.0 | 77° F. | 0 | 8816 |

### Example 3

6 cSt PAO was treated over a 100 cc volume of 13X molecular sieve, in a glass column, bed at temperatures from 50° F. to 95° F. and at liquid space velocities ranging from 0.6 to 3.0 hours. Table-5 compares Molecular Sieve treatment to the industry accepted steam distillation for odor removal.

TABLE 5

| Product | Treatment | Odor Level |
|---|---|---|
| 6 cSt PAO | None | 5 (strong odor) |
| 6 cSt PAO | Steam distillation. | 0 (No odor) |
| 6 cSt PAO | Molecular Sieve | 0 (No odor) |

Table-6 compares the adsorption capacity of 13X molecular sieves as a function of residence time for 6 cSt PAO. The adsorption capacity increases with the higher residence time.

TABLE 6

| | LHSV | | Odor | Bed Life: Grams |
|---|---|---|---|---|
| Product | hours | Temperature | Level | Of Oil Processed |
| 6 cSt PAO | 3.0 | 77° F. | 0 | 765 |
| 6 cSt PAO | 1.8 | 77° F. | 0 | 1320 |
| 6 cSt PAO | 0.6 | 77° F. | 0 | 2400 |

TABLE 7
6 cSt Deodorization Using 13X Molecular Sieve Packed Column

| | | | | Oil | | Ratio |
|---|---|---|---|---|---|---|
| | | | | Absorbed | | PAO:Mol |
| | | Total | | in Molecular | | Sieve |
| | Temp | Flow | LHSV | Sieve | % | Weight |
| Run # | F. | gms | (hours) | (grams) | Loss | Basis |
| PureSyn ® 6* | | | | | | |
| 1 | 95 | 1638 | 0.6 | 50.9 | 3.1 | 27.25 |
| 2 | 77 | 2263 | 0.6 | 46.9 | 2.1 | 38.1 |
| 3 | 50 | 2564 | 0.6 | 29.6 | 1.15 | 41.0 |

*PureSyn ® 6 is a C10 based 6cSt PAO sold by ExxonMobil Chemical.
Note:
Run numbers 1 and 2 could have been extended to give total throughput close to 2500 grams. We believe that these experiments were prematurely terminated. The end of run is when a distinct odor is detected on repeated smelling.

TABLE 8
2 cSt Deodorization Using 13X Mol Sieve Packed Column

| | | | | | | Ratio | |
|---|---|---|---|---|---|---|---|
| | | | | Oil | | PAO | |
| | | | | Absorbed | | Mol | |
| | | Total | | in Mol | | Sieve | |
| Run | Temp. | Flow | | Sieve | % | Weight | |
| # | ° F. | gms | LHSV | gms | Loss | Basis | Comments |
| 1 | 95 | 9360 | 3.0 | 33.0 | 0.35 | 139.7 | |
| 2 | 77 | 8816 | 3.0 | 33.2 | 0.37 | 137.8 | |
| 3 | 50 | 8500 | 3.0 | 33.0 | 0.39 | 133.0 | |
| 4 | 77 | 8696 | 3.0 | 25.0 | 0.29 | 127.5 | One |
| | | | | | | | regenera- |
| | | | | | | | tion |
| 5 | 77 | 8326 | 3.0 | 28.0 | 0.33 | 128.0 | Two |
| | | | | | | | regenera- |
| | | | | | | | tions |
| 6 | 77 | 8300 | 3.0 | 27.8 | 0.33 | 126.0 | Three |
| | | | | | | | regenera- |
| | | | | | | | tions |

TABLE 9

| | | | | Bed Life: | Ratio |
|---|---|---|---|---|---|
| | | | | Grams of | PAO:Mol |
| | LHSV | | Odor | Oil | Sieve |
| Product | (hours) | Temperature | Level | Processed | Weight Basis |
| 2 cSt PAO | 6.0 | 77° F. | 0 | 6029 | 97:1 |
| 2 cSt PAO | 3.0 | 77° F. | 0 | 8816 | 142:1 |
| 2 cSt PAO | 1.5 | 77° F. | 0 | 12300 | 198:1 |

TABLE 10

| | | | | Bed Life: | |
|---|---|---|---|---|---|
| | | | | Grams of | Ratio PAO |
| | LHSV | | Odor | Oil | Mol Sieve |
| Product | (hours) | Temperature | Level | Processed | Weight Basis |
| 4 cSt PAO | 3.0 | 77° F. | 0 | 900 | 14.5:1 |
| 4 cSt PAO | 1.8 | 77° F. | 0 | 3400 | 55:1 |

TABLE 11

| | | | | Bed Life: | Ratio |
|---|---|---|---|---|---|
| | | | | Grams of | PAO:Mol. |
| | LHSV | | Odor | Oil | Sieve |
| Product | (hours) | Temperature | Level | Processed | Weight Basis |
| 6 cSt PAO | 3.0 | 77° F. | 0 | 765 | 12.3:1 |
| 6 cSt PAO | 1.8 | 77° F. | 0 | 1320 | 21:1 |
| 6 cSt PAO | 0.6 | 77° F. | 0 | 2400 | 39:1 |

TABLE 12
Effect of In-Situ Regeneration On Bed Life
2 cSt PAO*

| | | | | Oil Ab- | | Ratio | |
|---|---|---|---|---|---|---|---|
| | | | | sorbed | | PAO:Mol | |
| | | Total | | in Mol | | Sieve | |
| Run | Temp. | Flow | | Sieve | % | Weight | Com- |
| # | ° F. | (Grams) | LHSV | Gms | Loss | Basis | ments* |
| 1 | 77° | 8816 | 3.0 | 33.2 | 0.37 | 137.8 | Base |
| | | | | | | | Case |
| 2 | 77° | 8696 | 3.0 | 25.0 | 0.29 | 127.5 | One |
| | | | | | | | regen- |
| | | | | | | | eration |
| 3 | 77° | 8326 | 3.0 | 28.0 | 0.33 | 128.0 | Two |
| | | | | | | | regen- |
| | | | | | | | erations |
| 4 | 77° | 8300 | 3.0 | 27.8 | 0.33 | 126.0 | Three |
| | | | | | | | regen- |
| | | | | | | | erations |

*(Regeneration At 650° F. and full vacuum)

We claim:

1. A method for removing odor-causing species from a lubricant composition, which comprises contacting said composition with at least one zeolite.

2. The method of claim 1, wherein said zeolite is a Type 4A or Type 13X Molecular Sieve.

3. The method of claim 1, wherein said zeolite is a Type 13X Molecular Sieve.

4. The method of claim 2, wherein said Molecular Sieve has been pre-treated by heating at a temperature of at least about 300° F. and a pressure of less than about 3 psig for a period of time sufficient to remove traces of water.

5. The method of claim 1, wherein said lubricant composition is comprised of a 2-10 cSt poly α-olefin derived lubricant.

6. A method for removing odor-causing species from a lubricant composition which comprises feeding said lubricant to a column packed with Type 13X Molecular Sieve at a temperature of about 40° F. to 130° F., at a pressure of about 0 psig to 5 psig, for an average residence time of about 0.15 hours to 3.5 hours.

7. The method of claim 6, wherein said lubricant composition is comprised of at least one poly α-olefin derived lubricant having a viscosity of from about 2-10 cSt at 100° C.

8. The method of claim 6, wherein the pressure is about 0 psig to about 3.0 psig.

9. The method of claim 6, wherein the residence time is about 1 hour to about 3.5 hours.

10. The method of claim 6, wherein the temperature is about 70-100° F.

11. The method of claim 6, wherein the temperature is about 70 to 95 F and the residence time is abut 1.5-2.0 hours.
